(19) United States
(12) Patent Application Publication
(10) Pub. No.: US 2024/0189581 A1
(43) Pub. Date: Jun. 13, 2024

(54) MEDICAL DEVICE IMPLANT TEMPLATE SYSTEMS AND METHODS

(71) Applicant: Shiratronics, Inc., Brooklyn Park, MN (US)

(72) Inventors: Scott A. Spadgenske, Buffalo, MN (US); Alan H. Smythe, White Bear Lake, MN (US)

(21) Appl. No.: 18/532,206

(22) Filed: Dec. 7, 2023

Related U.S. Application Data
(60) Provisional application No. 63/431,779, filed on Dec. 12, 2022.

(51) Int. Cl. A61N 1/05

(52) U.S. Cl. CPC A61N 1/0526; A61N 1/0504

(57) ABSTRACT

A system may include a medical device template for use to implant a medical device having a bottom surface with a periphery and a contour to follow an anatomical feature. The template may include a bottom surface with a periphery and a contour that correspond to the periphery and the contour of the bottom surface of the medical device. The template may be configured to be positioned and orientated over the anatomical feature to facilitate marking skin over the anatomical feature used to determine where to make an incision to implant the medical device. The system may include a frame configured to be worn on a head, with adjustable attachments configured for use to bilaterally position and orientate the templates against the head. The medical device may be configured to be subcutaneously implanted over a skull. The contour may correspond to a contour of the skull.

### PRIORITY

This application claims the benefit of priority to U.S. Provisional Application Ser. No. 63/431,779, filed Dec. 12, 2022, which is incorporated by reference herein in its entirety.

### TECHNICAL FIELD

This document relates generally to medical systems, and more particularly, but not by way of limitation, to systems, devices, and methods to implant medical device(s) using template(s).

### BACKGROUND

Medical devices may include implantable devices configured to deliver a therapy, such as but not limited to an electrical therapy and/or to sense electrical signals to detect physiological or functional parameters or other health-related data. Implantable neurostimulators are a specific example of implantable electrical therapy devices. A fully head-located implantable peripheral neurostimulation system, having one or more implantable devices, designed for the treatment of chronic head pain is a specific example of an implantable neurostimulation system.

Human anatomy is variable from patient-to-patient which can make it difficult to select site(s) for implanting medical device(s). For example, fully head-located implantable peripheral neurostimulation systems may be subcutaneously implanted in patient heads that can vary significantly in shapes and sizes.

Improved systems, devices and methods are needed to assist clinicians with selecting site(s) for implanting medical device(s). This may be particularly desirable for selecting sites for multiple medical devices including situations in which it is desirable to maintain symmetric implant locations.

### SUMMARY

An example (e.g., “Example 1”) of a method for implanting at least one medical device may include locating at least one reference anatomical feature, and placing at least one medical device template, based on the at least one reference anatomical feature, on a surface over at least one location where the at least one medical device is to be implanted. The medical device template(s) may have a bottom surface to contact the surface and the medical device(s) may have a bottom surface. The template bottom surface and the device bottom surface may have matching peripheries and contours. The method may further include marking where the at least one medical device template is placed on the skin surface, and implanting the at least one medical device at the at least one location using the marking as a guide.

In Example 2, the subject matter of Example 1 may optionally be configured such that placing the at least one medical device template includes orientating the medical device template over the at least one location where the at least one medical device is to be implanted.

In Example 3, the subject matter of any one or more of Examples 1-2 may optionally be configured such that the at least one medical device template is adjustably attached to a frame. Placing the at least one medical device template may include placing the frame on a subject and adjusting the at least one medical device template on the frame.

In Example 4, the subject matter of any one or more of Examples 1-3 may optionally be configured such that the at least one location includes a location in a head.

In Example 5, the subject matter of Example 4 may optionally be configured such that the at least one location includes a subcutaneous location over a skull in the head.

In Example 6, the subject matter of any one or more of Examples 4-5 may optionally be configured such that the at least one location includes a location in the face.

In Example 7, the subject matter of any one or more of Examples 4-6 may optionally be configured such that the at least one location includes a bilateral location.

In Example 8, the subject matter of any one or more of Examples 1-7 may optionally be configured such that locating at least one reference anatomical region includes locating an inion on a skull, and the at least one medical device template is placed based on a location of the inion.

In Example 9, the subject matter of any one or more of Examples 1-8 may optionally be configured such that locating at least one reference anatomical region includes locating a nuchal ridge, and the at least one medical device template is placed above the nuchal ridge.

An example (e.g., “Example 10”) of a method may include placing a frame with a first medical device template and a second medical device template on a head. Each of the at least two medical device templates may have a bottom surface to contact a surface of the head and the at least one medical device may have a bottom surface that has a periphery. The method may include adjusting a position of the first medical device template to be on a left side of the head and superior to a nuchal ridge on the left side of the head, and adjusting a position of the second medical device template to be on a right side of the head and superior to a nuchal ridge on the right side of the head. The adjusted positions of the first and second medical device templates may be an equal distance from an inion and symmetrical to a midline passing through the inion. The method may include marking the locations of both the first and second medical device templates on the surface of the head, making a first incision through skin on the head superior to the location of the first medical device template and making a second incision through the skin on the head superior to the location of the second medical device template. The method may further include inserting a first medical device through the first incision in an inferior direction into a first pocket beneath the skin toward the nuchal ridge on the left side of the head, and inserting a second medical device through the second incision in an inferior direction into a second pocket beneath the skin toward the nuchal ridge on the right side of the head. Each of the first and second medical devices may have a bottom surface with a periphery that corresponds to the peripheries of the bottom surface of the first and second medical device templates.

In Example 11, the subject matter of Example 10 may optionally be configured such that the bottom surface for each of the first and second medical devices has a contour, and the bottom surface for each of the first and second medical device templates has a contour corresponding to the contour of the bottom surface for the first and second medical devices.

In Example 12, the subject matter of Example 11 may optionally be configured such that the contour of the bottom surface for the first and second medical devices is designed to follow a contour of the surface of the head.

In Example 13, the subject matter of Example 12 may optionally be configured to further include adjusting an orientation of the first and second medical device templates to cause the contour of the bottom surface for each of the first and second medical device templates to closely follow the contour of the head at the marked locations.

In Example 14, the subject matter of any one or more of Examples 10-13 may optionally be configured to further include, for each of the first and second implantable medical devices, subcutaneously inserting a first lead to stimulate a greater occipital nerve and subcutaneously inserting a second lead to stimulate a supraorbital nerve.

An example (e.g., “Example 15”) of a system may include a medical device template for use to implant a medical device having a bottom surface with a periphery and a contour to follow an anatomical feature. The medical device template may include a bottom surface with a periphery and a contour that correspond to the periphery and the contour of the bottom surface of the medical device. The medical device template may be configured to be positioned and orientated over the anatomical feature to facilitate marking skin over the anatomical feature used to determine where to make an incision to implant the medical device.

In Example 16, the subject matter of Example 15 may optionally be configured such that the bottom surface of the medical device has at least a first plane and a second plane that form an obtuse angle such that the contour of the bottom surface is at least partially defined by the first plane, the second plane and the obtuse angle.

In Example 17, the subject matter of any one or more of Examples 15-16 may optionally be configured to further include a frame configured to be positioned on a subject. The frame may include an adjustable attachment for attaching to the medical device. The adjustable attachment of the frame may be configured to enable the medical device to be repositioned and reoriented with respect to the frame that is positioned on the subject.

In Example 18, the subject matter of Example 17 may optionally be configured such that the medical device template is a first medical device. The system may further include at least one additional medical device template including a second medical device template. The frame may include corresponding adjustable attachments for each of the at least one medical device template.

In Example 19, the subject matter of Example 18 may optionally be configured such that the frame is configured to be worn on a head. The frame and the adjustable attachments may be configured for use to bilaterally position and orientate the medical device templates against the head symmetrically to a midline passing through an inion and above a nuchal ridge.

In Example 20, the subject matter of any one or more of Examples 15-19 may optionally be configured such that the medical device is configured to be subcutaneously implanted over a skull, and the contour of the bottom surface corresponds to a contour of the skull.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate a system that includes implantable medical device(s) and an external device configured for use to communicate with and charge the implantable medical device(s).

FIG. 2A depicts two implanted devices with leads to cover both sides of the head with one of the devices on the left side of the head and the other on the right side of the head, and FIG. 2B illustrates a charging/communication headset disposed about the cranium.

FIGS. 3A-3C illustrate, by way of example and not limitation, an implantable medical device.

FIG. 4 illustrate a headset 403, similar to the headset illustrated in FIGS. 1A-1B.

FIG. 5 illustrates, by way of example and not limitations, medical device templates adjustably attached to a frame.

FIG. 6A illustrates a side view of an implantable medical device and FIG. 6B illustrate a device bottom surface of the implantable medical device.

FIG. 7A illustrates a side view of a medical device template, and FIG. 7B illustrate a template bottom surface for the medical device template.

FIG. 8 illustrates, by way of example and not limitation, a side view of fully head-located neurostimulation system.

FIGS. 9A-9B illustrate, by way of example and not limitation, a technique to find a desirable location to implant the implantable medical device to stimulate the greater occipital nerve and the supraorbital nerve.

FIG. 10 illustrates, by way of example and not limitation, a system for bilaterally positioning and orientating medical device templates for use in subcutaneously implanting neurostimulators over the cranium.

FIG. 11 illustrates a method for implanting at least one medical device.

FIG. 12 illustrates a method that includes placing a frame with a first medical device template and a second medical device template on a head.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

This present subject matter supports the implantation of an implantable medical device at a desirable location and orientation. For example, the medical device may be configured to be subcutaneously implanted beneath the skin and over bone, muscle, cartilage or other tissue. Some embodiments of the present subject matter support the implantation of more than one medical device.

The present subject matter uses one or more medical device templates that enable a clinician to visualize the size, location and/or orientation of implants before making incisions. The system is capable of accommodating varying anatomy, such as head anatomy. The intended implant location for each medical device may be a subcutaneous location in a head, such as a location beneath the skin and over the cranium. The human head varies in shape and size varies from patient to patient, and features of the human head may vary between the left and right sides of head. For example, it may be difficult to maintain symmetry about a midline of the patient for bilaterally-implanted multiple head-located implantable neurostimulators.

A medical device may be designed with a contour to follow a curvature of patient anatomy, such as but not limited a curvature of the cranium. For example, for fully head-located medical devices to be subcutaneously implanted, it is desirable for the device to have a low profile for reasons associated with patient conform and cosmetics. Therefore, a bottom surface of the medical device may be designed with a contour to closely fit the curvature of the head (e.g., cranium or face) and a desired implant location. According to various embodiments, a contour and/or periphery of the template(s) may correspond to the contour and/or periphery of the medical device such that the position and orientation of the template over the skin positions the contour of the template to also follow the curvature of the patient anatomy. The corresponding contours and/or peripheries may be exact matches or may be similar contours and/or peripheries and not necessarily an exact match. The contour and/or periphery of the bottom surface of the template emulates the contour and/or periphery of the bottom surface of the medical device for visualization of a desired position and orientation of the implanted medical device.

System(s) for placing template(s) may be capable of three-dimensional movement allowing a clinician to manipulate each template so that the clinician can visualize and identify implant locations and incision sites. For example, a clinician may use anatomic landmarks and locate nerve pathways during an implantation of a subcutaneous chronic migraine system, and may mark skin to ensure visualization of anatomical landmarks, identification of nerve pathways, and planning of lead placement path. By way of example, a clinician may identify the nuchal ridge, inion, a supraorbital line for a lead path, an occipital line for a lead path, and other desired landmarks. The template(s) and markings allow the clinician to visualize the intended location of the lead pathways with respect to the landmarks, and make markings as desired. Once a desirable location is selected using the template, the clinician may appropriately prep the patient for the procedure with potential removal of hair, marking device location, marking incision location, and performing other perioperative surgical preparation. Determining and marking the implant and incision site helps simplify the implant procedure and reduce the risk of implanting the device(s) at an incorrect location. For example, various embodiments may use a frame, such as a headset, with adjustably attached medical device templates that emulate actual implantable devices. The templates may be on a movable arm, which may accommodate movement in x, y, and z directions. The orientation of the templates, which may have a contour on a bottom surface to correspond to a curvature of the anatomy, may also be adjusted. Thus, the clinician may use a fit of the implant over the skin to approximate how the medical device will fit to the curvature of the cranium beneath the implant.

The template(s) simplify the procedure and reduce risks associated with making an incorrect incision location for insertion of the medical device. It is desirable for the template(s) to be comfortable for the patient to wear or to otherwise be applied to the patient. It is also desirable for the system to enable a clinician to easily adjust and configure implant locations.

Template(s) may be used to visualize and plan various medical device implant procedures before actually implanting the device. As the template(s) rest against or near the skin surface of the patient, it is believed that the template(s) may be particularly useful for planning subcutaneous implant procedures. The template(s) may also be useful to provide cosmetically-acceptable implant sites for visible implant locations. This disclosure will discuss, by way of example and not limitation, the use of templates with a fully head-located neurostimulation system. As such, an example of a fully head-located neurostimulation system is described below. However, the template(s) may be used with other implantable medical device(s).

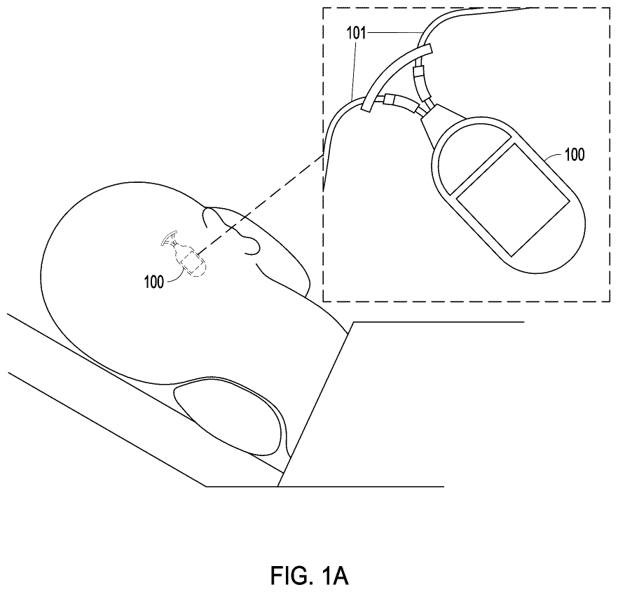

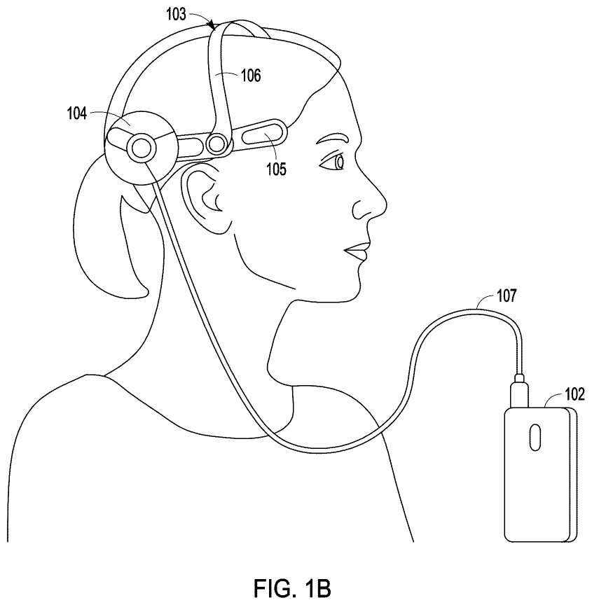

FIGS. 1A-1B illustrate a system that includes implantable medical device(s) and an external device configured for use to communicate with and charge the implantable medical device(s). More particularly, the system is an example of a fully head-located neurostimulation system designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias.

FIG. 1A illustrates an implantable medical device 100 implanted beneath the skin and over a patient's cranium. The device 100 is illustrated as being implanted behind and above the ear. The implantable medical device 100 may include one or more leads 101 that may be subcutaneously tunneled to a desired neural target. Each lead may include one or more electrodes. The number of electrodes and spacing may be such as to provide therapeutic stimulation over any one or any combination of the supraorbital, parietal, and occipital region substantially simultaneously. The implantable medical device 100 may be configured to independently control each electrode to determine whether the electrode will be inactive or configured as an electrode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 1B illustrates an external device 102 and headset 103 configured for use to communicate with and/or charge the implantable medical device(s) 100. The headset 103 may include an external coil 104, and the headset 103 may be configured to position the external coil over an implantable medical device. For example, the headset 103 may include an adjustable frame 105 on each side of the head that can rotate about a point on a main headset frame 106, and may be configured to provide addition degrees of motion (e.g., sliding or pivoting motion) with respect to the main headset frame 106. These adjustable frames may be used to position the external coils 104 over the implantable medical devices 100 when the main headset frame 106 is worn. The external device 102 may be electrically connected to the external coil 104 via a cable 107. In some embodiments, the external device 102 may be wirelessly connected to the headset 103. The headset may be configured to wirelessly receive power from the external device and to transfer power from the external coil to the implanted device(s).

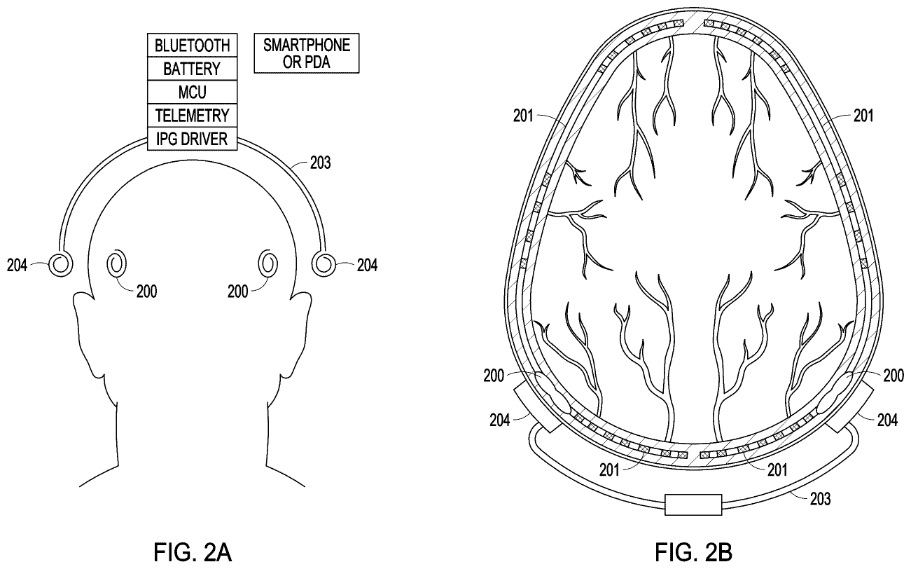

FIG. 2A depicts two implanted devices 200 with leads 201 to cover both sides of the head with one of the devices on the left side of the head and the other on the right side of the head, and FIG. 2B illustrates a charging/communication headset 203 disposed about the cranium. The headset 203 may include right and left coupling coil enclosures, respectively that contain coils for coupling to the respective coils in the implants. The coil enclosures may interface with a main charger/processor body which may contain processor circuitry and batteries for both charging the internal battery in the implantable medical devices 200 and also communicating with the implanted devices. Thus, in operation, when a patient desires to charge their implanted devices 200, all that is necessary for some embodiments is to place the headset about the cranium with the coils 204 in close proximity to the respective implanted devices 200. In some embodiments, such placement may automatically initiate charging; whereas in other embodiments, the user may initiate charging using an external device. When the headset 203 is worn by a patient, the headset coils (transmit coils) 204 are placed in proximity to the corresponding receive coil in each respective body-implanted implantable medical device 200. As illustrated, the headset 203 may include an implantable medical device driver, telemetry circuitry, a controller, a battery, and a Bluetooth wireless interface. The headset 203 may also communicate with a personal device such as a smartphone or tablet (e.g., via the Bluetooth interface), for monitoring and/or programming operation of the two implantable medical devices.

The implantable medical device 200 may include a rechargeable battery, an antenna (e.g., coil), and at least one ASIC, along with the necessary internal wire connections amongst these related components, as well as to the incoming lead internal wires. These individual components may be encased in a can made of a medical grade metal, which may be encased by plastic cover. The battery may be connected to the ASIC(s) via a connection that is flexible. The overall enclosure for the battery, antenna and ASIC(s) may have a very low flat profile with two lobes, one lobe for housing the ASIC(s) and one lobe for housing the battery. The antenna may be housed in either of the lobes or in both lobes. The use of the two lobes and the flexible connection between the ASIC(s) and the battery allows the implanted device to conform to the shape of the human cranium when subcutaneously implanted without securing such to any underlying structure with an external fixator.

The ASIC(s) and lead may be configured to independently drive each of the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular programs to provide the necessary therapeutic stimulation. The ASIC(s) may have memory and be configured for communication and for charge control when charging a battery. Each of the set of wires and interface with the ASIC(s) such that the ASIC(s) individually controls each of the wires in the particular bundle of wires. Thus, each electrode may be individually controlled. Each electrode may be individually turned off, or as noted above, each electrode can be designated as an anode or a cathode. During a charging operation, the implanted device is interfaced with an external charging unit via the antenna (e.g., coil) which is coupled to a similar antenna (e.g., coil) in the external charging unit. Power management involves controlling the amount of charge delivered to the battery, the charging rate thereof and protecting the battery from being overcharged.

The ASIC(s) may be capable of communicating with an external unit, typically part of the external charging unit, to exchange information. Thus, configuration information can be downloaded to the ASIC and status information can be retrieved. Although not illustrated herein, a headset or the like may be provided for such external charging/communication operation.

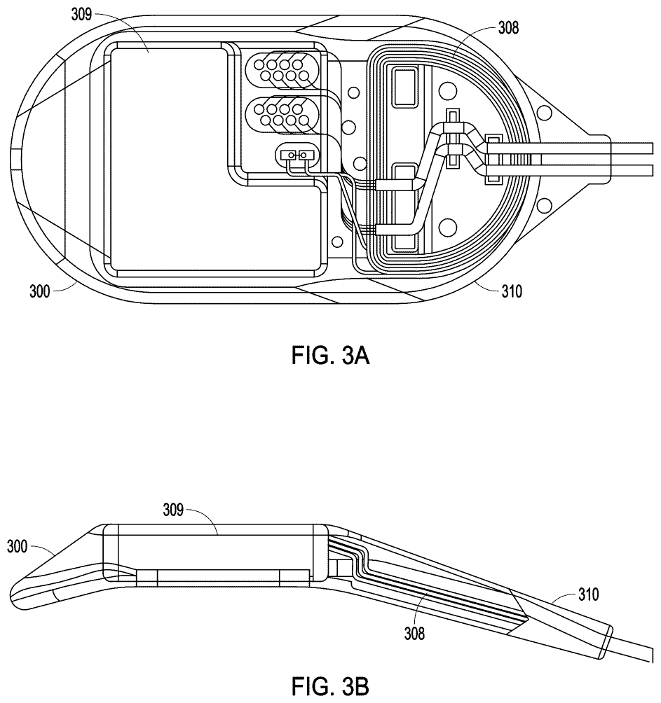

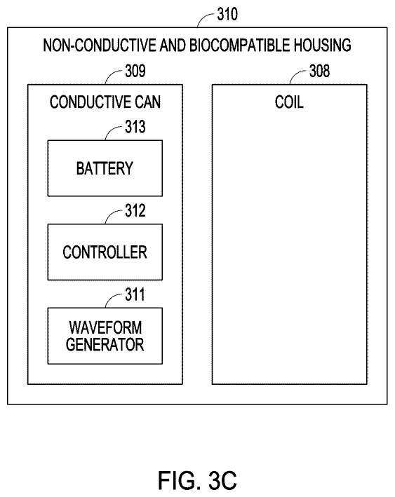

FIGS. 3A-3C illustrate, by way of example and not limitation, an implantable medical device. The illustrated device 300 is configured for subcutaneous implantation under the skin but over bone, muscle or other tissue such as cartilage. For example, the illustrated device may be subcutaneous implanted over a cranium. The device 300 includes a coil 308 used for communication and charging, a conductive enclosure 309 (e.g., metal can) for electronics, and a non-conductive and biocompatible coating 310 that encases the conductive enclosure 309 and the coil 308. The electronics within the conductive enclosure may include a neurostimulator waveform generator 311, a controller 312 and a battery 313. At least one microcontroller unit (MCU) and/or at least one application specific integrated circuit (ASIC) may provide the controller and the neurostimulator waveform generator functions. The MCU(s) may contribute to one or both of the controller and the neurostimulator waveform generator functions. The ASIC(s) may contribute to one or both of the controller and the neurostimulator waveform generator functions. The MCU(s) and/or ASIC(s) may be encased within the conductive enclosure. The coil is not within the conductive enclosure, so that it can be used to perform charging and communication functions using an electromagnetic field. The non-conductive housing may include silicone or an epoxy. Also, the non-conductive housing is a poor conductor of heat, and thus insulates the tissue from any heat generated at the coil or the conductive enclosure. The housing may be flexible, allowing some motion or flex when implanted, which encourages a low profile as it follows a curvature of a cranium when subcutaneously implanted over the cranium.

The illustrated housing may include a first housing portion and a second housing portion, where the first housing portion encapsulates the coil and the second housing portion encapsulates the conductive housing (e.g., metal can). The first and second housing portions may have substantially equal footprints. Each of the first and second housing portions have a thickness, length and width. The thickness may be uniform and may be less than the length and the width. Each of the first and second housing portions may have a substantially planar major surface, wherein the first and second housing portions are joined such that the substantially planar major surfaces form an angle between 90 degrees and 180 degrees. This angle allows the substantially planar major surfaces for the first and second housing portions to follow the curvature of the cranium. Another lobe may be included, which may encourage the implanted device to remain in place when implanted as it provides the implantable medical device with a profile that follows the curvature of the cranium.

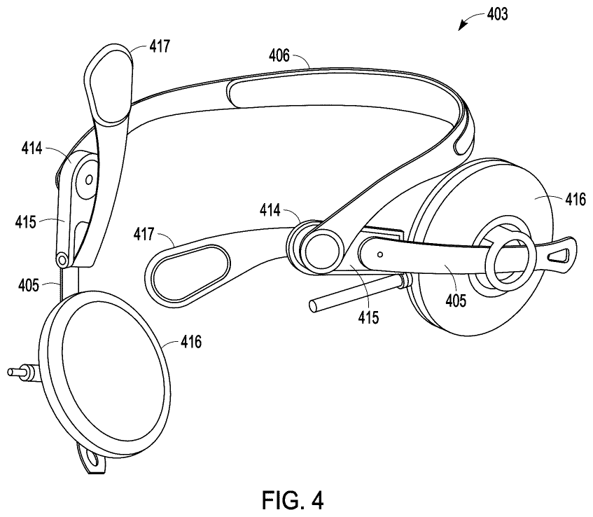

FIG. 4 illustrate a headset 403, similar to the headset illustrated in FIGS. 1A-1B. The headset 403 may include an adjustable frame 405 on each side of a main headset frame 406. The adjustable frames 405 may be rotatably connected at pivot points 414 to the main headset frame 406. Each adjustable frame 405 may include a secondary pivot point 415 to enable further adjustment of the coil enclosure 416 for placement over the head-located implanted devices. A bowed end 417 of the adjustable frames, opposite to the end with the coil enclosure 416, may be configured with bend inward to gently press against the head.

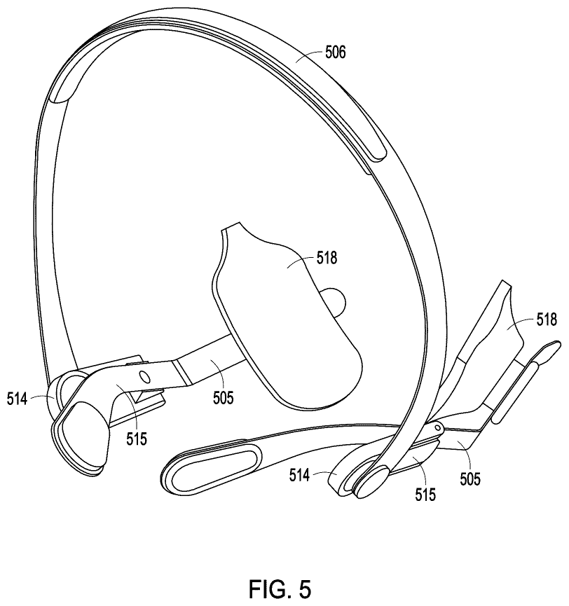

FIG. 5 illustrates, by way of example and not limitations, medical device templates adjustably attached to a frame. The illustrated embodiment uses a similar main headset frame 506 attached to adjustable frames 505 at pivots points 514 and 515. The coil enclosures 416 from the headset illustrated in FIG. 4 may be replaced with medical device templates 518. Other frame designs with adjustable attachment features may be implemented for use to position and/or orientate one or more medical device templates. The frame may enable different pivoting motion and sliding motion of the adjustable frames 505 as well as pivoting motions where the templates attach to the adjustable frames 505. Other frame designs may be used to provide three-dimensional movement of the templates and orientation adjustments to the templates.

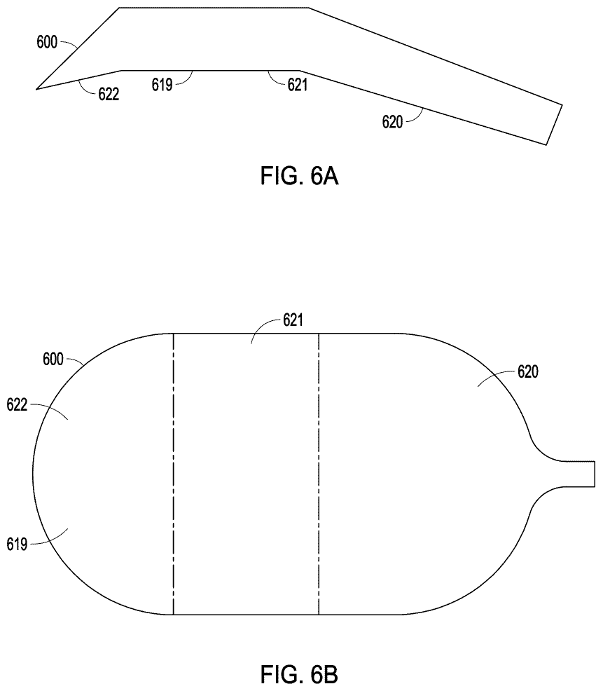

FIG. 6A illustrates a side view of an implantable medical device and FIG. 6B illustrate a device bottom surface of the implantable medical device. The implantable medical device 600 has a device bottom surface 619, which has a contour configured to contact anatomical structures below the skin. Examples of such anatomical structures include bone such as the cranium, muscle, cartilage, or other tissue. The contour of the device bottom surface 619 promotes a low profile of the medical device when subcutaneously over a curved surface such as the cranium. If the bottom surface was flat, rather than having a profile with at least two planes, and if the medical device was implanted over the curved surface such as the cranium, then the edges of the implantable device may push up on the skin, causing “tenting”. The illustrated embodiment has at least a first planar surface 620 and a second planar surface 621 to provide a generally concave profile. Some embodiments may include a third planar surface 622 that also contributes to the concave surface. Some embodiments of the medical device may be designed with a concave bottom surface that does not have planar surfaces.

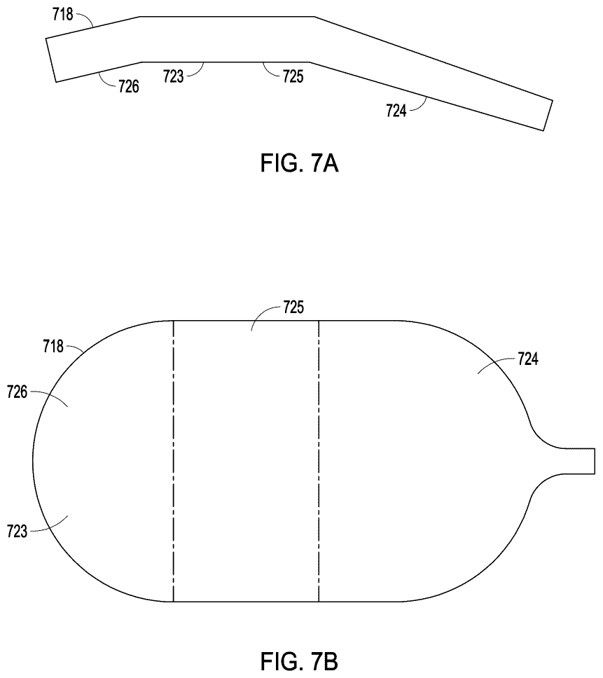

FIG. 7A illustrates a side view of a medical device template, and FIG. 7B illustrate a template bottom surface for the medical device template. The medical device template 718 has a template bottom surface 723. The periphery of the template bottom surface 723 generally corresponds to the periphery of the device bottom surface 619 such that the template allows the clinician to visualize the size and location of the later-implanted medical device 600. In some embodiments, the peripheries exactly or approximately match. However, the template may be designed so that the bottom surface does not have a periphery that is an exact match to the periphery of the medical device. For example, the template may have a feature extending out to facilitate marking an incision location that will be outside of a footprint of the medical device. The medical device may be slid through the incision into a pocket under the skin where the template was located. The template bottom surface 723 also has a contour which exactly or approximately matches the contour of the device bottom surface. For example, the template bottom surface 723 may have a first planar surface 724, a second planar surface 725 and a third planar surface 726. Similar to the device bottom surface 619, the template bottom surface 723 as a concave profile. In some embodiments, the contour of the device bottom surface 619 and the template bottom surface 723 are exactly the same. In some embodiments, the contour of the template bottom surface 723 approximates the contour of the device bottom surface 619, such as template bottom surface 723 having a smooth concave shape without planar surface to approximate the concave shape of the device bottom surface 619 with the planar surfaces. The same or similar contours of the bottom surfaces allow the template to be both positioned and oriented over the skin in a manner that provides a good fit of the contour to the anatomy. This assists the clinician with visualizing how the medical device will fit over bone (e.g., cranium) or other tissue when implanted.

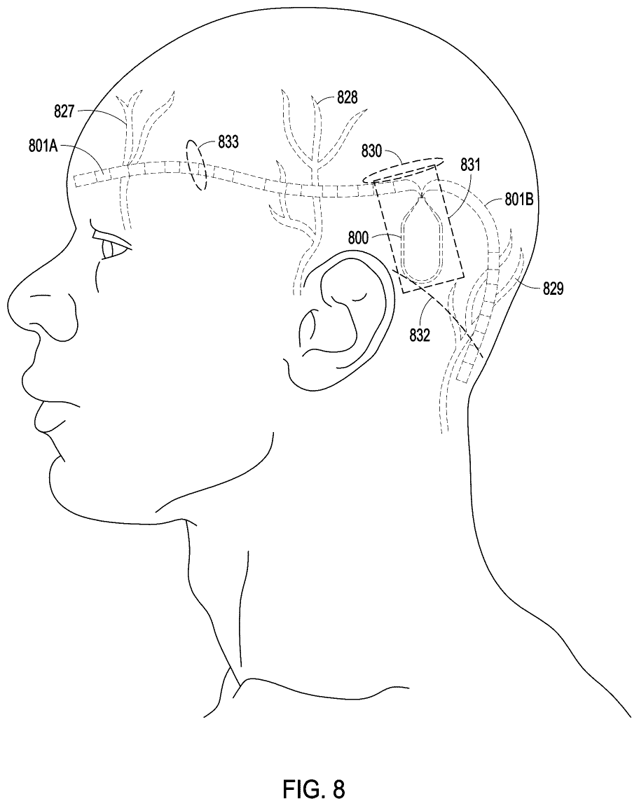

FIG. 8 illustrates, by way of example and not limitation, a side view of fully head-located neurostimulation system. Also illustrated are the supraorbital nerve 827, the auriculotemporal nerve 828 and the occipital nerve 829. The illustrated system includes a medical device (e.g., pulse generator) 800 and both a first lead 801A and a second lead 801B attached to the medical device 800. The medical device 800 and leads 801A and 801B are configured for use in stimulating the supraorbital nerve 827 toward the front of the head and the occipital nerve 829 toward the back of the head.

The patient may have had a period of trial neurostimulation, which is standard in traditional neurostimulator evaluations but is optional here. The actual permanent implant may occur in a standard operating suite with appropriate sterile precautions. By way of example and not limitation, the patent may be prepped and draped. The patient may be administered prophylactic antibiotics, local anesthetic, and sedation. The patient may be placed in a supine position with a head of the bed elevated to approximately thirty degrees. The patient's head may be turned to better access the intended implant location. While the implantable medical device may be positioned subcutaneously anywhere, it may be positioned above and behind the ear in this illustrated embodiment. Thus, a first incision 830 of sufficient length (approximately 4-6 cm) is made to a depth sufficient to reach the subcutaneous layer. A pocket 831 to accept the medical device 800 is fashioned by standard dissection techniques. The pocket 831 may be directed below the incision. The pocket 831 may be angled depending on the desired orientation of the medical device. For example, the pocket 831 may be angled posteriorly, as illustrated. The pocket 831 may be 10-20% larger than the medical device 800 to allow for a comfortable fit and no undue tension on the overlying skin and/or incision. The first incision 830 may be made and the pocket 831 formed so that the implantable medical device abuts against the nuchal ridge 832 when fully inserted into the pocket 831. The first incision 830 should not interfere with the implanted medical device 800. The present subject matter may use template(s) to help make the incision in a desired location.

A second incision 833 may be made to the subcutaneous layer at a point above and anterior to the pinna of the ear in the temple region to assist with subcutaneously routing the first lead 801A. The first lead 801A may be passed from the medical device 800 in the pocket 831 to the first incision 830, and then passed from the first incision 830 to its final subcutaneous position over supraorbital nerves 827. The second lead 801B may be passed from the medical device 800 in the pocket 831 back toward the occipital nerve 829. The medical device 800 may be inserted into the pocket 831 either before or after the leads 801A and/or 801B are tunneled to their final subcutaneous position to deliver therapy.

Tubular introducer(s) with a plastic-peel away shell may be used to assist with lead placement. However, other techniques may be used to subcutaneously tunnel the leads to their final placement to deliver the neurostimulation therapy. Following the entire placement of the complete system, including the medical device and both leads and suturing, the medical device may be powered-up and its circuits checked. Upon recovery from anesthesia the system may be turned on for the patient with a portable programmer and the multiple parameters for the system may be programmed to provide a desired therapy for the patient.

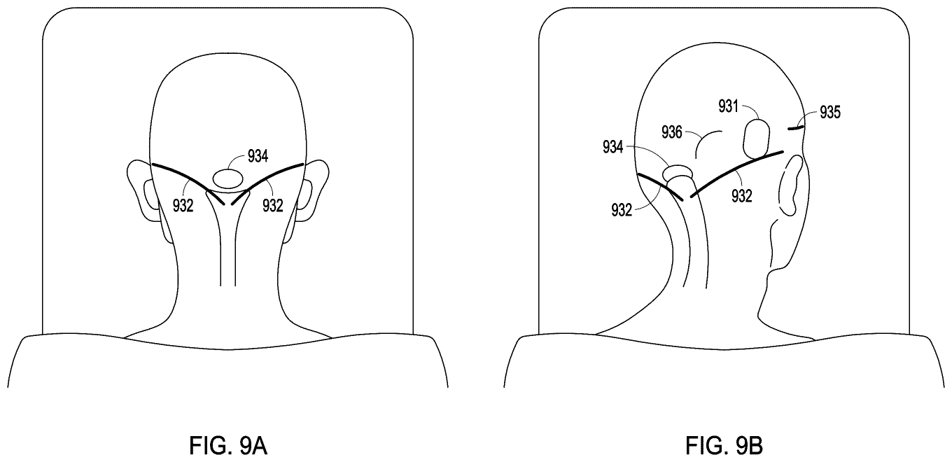

FIGS. 9A-9B illustrate, by way of example and not limitation, a technique to find a desirable location to implant the implantable medical device to stimulate the greater occipital nerve and the supraorbital nerve. A clinician may identify anatomical landmarks such as the inion 934 and the nuchal ridge 932. The inion 934 is the midline bony prominence in the occipital bone. It is located at the lower rear part of the human skull, and is usually easily perceptible to the clinician. The nuchal ridge 932 is on the occipital bone on the lower rear part of the skull where some neck muscles attach. It has been found that forming the pocket 931 to position the medical device just above the nuchal ridge 932 may be desirable as the nuchal ridge 932 discourages subcutaneous movement of the device. The implanted device cannot easily pass over the nuchal ridge 932.

Templates may be placed using the identified inion, 934 nuchal ridges 932 and/or other landmarks. The intended lead pathways (e.g., the supraorbital line 935 and/or the occipital line 936) from the templates may also be visualized and marked as desired. Once a desirable location is selected using the template, the clinician may appropriately prepare the patient for the procedure. For example, the clinician may remove hair and clean the surgical site, mark the device location, mark the incision location and/or perform other perioperative surgical preparation(s).

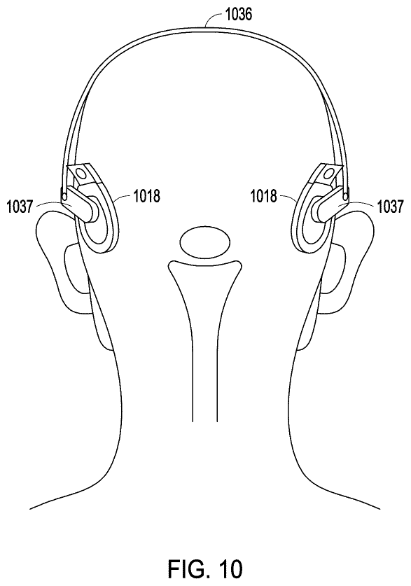

FIG. 10 illustrates, by way of example and not limitation, a system for bilaterally positioning and orientating medical device templates for use in subcutaneously implanting neurostimulators over the cranium. The illustrated system has a frame 1036, with adjustable attachments 1037 to each of two medical device templates 1018. Each of the adjustable attachments 1018 may include a pivot point and arm for use by the clinician to adjust a position of the template on the head. The adjustable attachment may include a mechanism to allow the attached template to pivot and at least partially rotate. Once in a desired location and/or orientation, the clinician may mark where the medical devices are to be implanted, the direction of the leads, and/or the incision line.

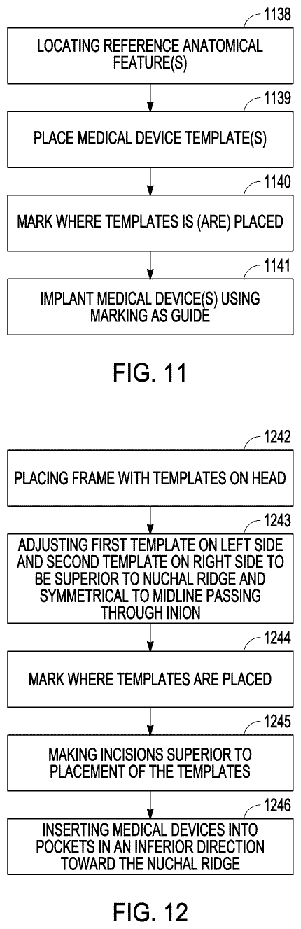

FIG. 11 illustrates a method for implanting at least one medical device. The method may include locating at least one reference anatomical feature 1138. Examples of anatomical features that may be used to implant a fully head-located neurostimulator include the inion and nuchal ridges. The method may further include placing at least one medical device template 1139, based on the at least one reference anatomical feature, on a surface over at least one location where the at least one medical device is to be implanted. The medical device template has a bottom surface to contact the surface and the medical device has a bottom surface. The template bottom surface and the device bottom surface may have similar or matching peripheries and contours. The method may further include marking where the at least one medical device template is placed on the skin surface 1140, and implanting the at least one medical device at the at least one location using the marking as a guide 1141.

FIG. 12 illustrates a method that includes placing a frame with a first medical device template and a second medical device template on a head 1242. Each of the at least two medical device templates has a bottom surface to contact a surface of the head and the at least one medical device having a bottom surface that has a periphery. The method may further include adjusting a position of the first medical device template to be on a left side of the head and superior to a nuchal ridge on the left side of the head, and adjusting a position of the second medical device template to be on a right side of the head and superior to a nuchal ridge on the right side of the head 1243. The adjusted positions of the first and second medical device templates may be an equal distance from an inion and symmetrical to a midline passing through the inion. The locations of both the first and second medical device templates on the surface of the head may be marked 1244. A first incision through skin on the head may be made superior to the location of the first medical device template and a second incision through the skin on the head may be made superior to the location of the second medical device template 1245. The method may further include inserting a first medical device through the first incision in an inferior direction into a first pocket beneath the skin toward the nuchal ridge on the left side of the head, and inserting a second medical device through the second incision in an inferior direction into a second pocket beneath the skin toward the nuchal ridge on the right side of the head 1246. Each of the first and second medical devices has a bottom surface with a periphery that corresponds to the peripheries of the bottom surface of the first and second medical device templates. The peripheries may be similar to each other or be exact matches of each other. Regardless, the corresponding peripheries allow a clinician to visualize the size and orientation of medical device(s) to be implanted based on the position of the template(s).

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for implanting at least one medical device, comprising:
locating at least one reference anatomical feature;
placing at least one medical device template, based on the at least one reference anatomical feature, on a surface over at least one location where the at least one medical device is to be implanted, the at least one medical device template having a bottom surface to contact the surface and the at least one medical device having a bottom surface, wherein the template bottom surface and the device bottom surface have matching peripheries and contours;
marking where the at least one medical device template is placed on the skin surface; and
implanting the at least one medical device at the at least one location using the marking as a guide.

2. The method of claim 1, wherein placing the at least one medical device template includes orientating the medical device template over the at least one location where the at least one medical device is to be implanted.

3. The method of claim 1, wherein the at least one medical device template is adjustably attached to a frame, wherein placing the at least one medical device template includes placing the frame on a subject and adjusting the at least one medical device template on the frame.

4. The method of claim 1, wherein the at least one location includes a location in a head.

5. The method of claim 4, wherein the at least one location includes a subcutaneous location over a skull in the head.

6. The method of claim 4, wherein the at least one location includes a location in the face.

7. The method of claim 4, wherein the at least one location includes a bilateral location.

8. The method of claim 1, wherein locating at least one reference anatomical region includes locating an inion on a skull, and the at least one medical device template is placed based on a location of the inion.

9. The method of claim 1, wherein locating at least one reference anatomical region includes locating a nuchal ridge, and the at least one medical device template is placed above the nuchal ridge.

10. A method, comprising:
placing a frame with a first medical device template and a second medical device template on a head, wherein each of the at least two medical device templates has a bottom surface to contact a surface of the head and the at least one medical device having a bottom surface that has a periphery;
adjusting a position of the first medical device template to be on a left side of the head and superior to a nuchal ridge on the left side of the head, and adjusting a position of the second medical device template to be on a right side of the head and superior to a nuchal ridge on the right side of the head, wherein the adjusted positions of the first and second medical device templates are equal distance from an inion and symmetrical to a midline passing through the inion;
marking the locations of both the first and second medical device templates on the surface of the head;
making a first incision through skin on the head superior to the location of the first medical device template and making a second incision through the skin on the head superior to the location of the second medical device template; and
inserting a first medical device through the first incision in an inferior direction into a first pocket beneath the skin toward the nuchal ridge on the left side of the head, and inserting a second medical device through the second incision in an inferior direction into a second pocket beneath the skin toward the nuchal ridge on the right side of the head,
wherein each of the first and second medical devices has a bottom surface with a periphery that corresponds to the peripheries of the bottom surface of the first and second medical device templates.

11. The method of claim 10, wherein the bottom surface for each of the first and second medical devices has a contour, and the bottom surface for each of the first and second medical device templates has a contour corresponding to the contour of the bottom surface for the first and second medical devices

12. The method of claim 11, wherein the contour of the bottom surface for the first and second medical devices is designed to follow a contour of the surface of the head.

13. The method of claim 12, further comprising adjusting an orientation of the first and second medical device templates to cause the contour of the bottom surface for each of the first and second medical device templates to closely follow the contour of the head at the marked locations.

14. The method of claim 10, further comprising, for each of the first and second implantable medical devices, subcutaneously inserting a first lead to stimulate a greater occipital nerve and subcutaneously inserting a second lead to stimulate a supraorbital nerve.

15. A system, comprising a medical device template for use to implant a medical device having a bottom surface with a periphery and a contour to follow an anatomical feature, wherein the medical device template includes a bottom surface with a periphery and a contour that correspond to the periphery and the contour of the bottom surface of the medical device, wherein the medical device template is configured to be positioned and orientated over the anatomical feature to facilitate marking skin over the anatomical feature used to determine where to make an incision to implant the medical device.

16. The system of claim 15, wherein the bottom surface of the medical device has at least a first plane and a second plane that form an obtuse angle such that the contour of the bottom surface is at least partially defined by the first plane, the second plane and the obtuse angle.

17. The system of claim 15, further comprising a frame configured to be positioned on a subject, the frame including an adjustable attachment for attaching to the medical device, wherein the adjustable attachment of the frame is configured to enable the medical device to be repositioned and reoriented with respect to the frame that is positioned on the subject.

18. The system of claim 17, wherein the medical device template is a first medical device, the system further comprising at least one additional medical device template including a second medical device template, wherein the frame includes corresponding adjustable attachments for each of the at least one medical device template.

19. The system of claim 18, wherein the frame is configured to be worn on a head, the frame and the adjustable attachments being configured for use to bilaterally position and orientate the medical device templates against the head symmetrically to a midline passing through an inion and above a nuchal ridge.

20. The system of claim 15, wherein the medical device is configured to be subcutaneously implanted over a skull, and the contour of the bottom surface corresponds to a contour of the skull.
